(19) United States
(12) Patent Application Publication
(10) Pub. No.: US 2011/0118180 A1
(43) Pub. Date: May 19, 2011

(54) METHOD OF TREATMENT OF DIABETES TYPE 2 COMPRISING ADD-ON THERAPY TO METFORMIN

(75) Inventors: Louise SILVESTRE, Paris (FR); Tianyue ZHOU, Hillsborough, NJ (US); Caroline SERT-LANGERON, Chatenay-Malabry (FR)

(73) Assignee: SANOFI-AVENTIS DEUTSCHLAND GMBH, Frankfurt am Main (DE)

(21) Appl. No.: 12/617,811

(22) Filed: Nov. 13, 2009

(51) Int. Cl. A61K 38/16; A61P 3/10; A61K 31/155

(52) U.S. Cl. 514/6.9; 514/635

(57) ABSTRACT

A method for the treatment of diabetes mellitus type 2 comprising administering
(c) desPro³⁶Exendin-4(1-39)-Lys₆-NH₂ or/and a pharmaceutically acceptable salt thereof, and
(d) metformin or/and a pharmaceutically acceptable salt thereof, to a subject in need thereof.

Subject of the present invention is a method for treatment of diabetes type 2 with AVE0010 (lixisenatide) as add-on therapy to administration of metformin.

Metformin is a biguanide hypoglycemic agent used in the treatment of Type 2 diabetes mellitus not responding to dietary modification. Metformin improves glycemic control by improving insulin sensitivity. Metformin is usually administered orally. However, control diabetes mellitus type 2 in obese patients by metformin may be insufficient. Thus, in these patients, additional measures for controlling diabetes mellitus type 2 may be required.

A first aspect of the present invention is a method for the treatment of diabetes mellitus type 2 comprising administering
(a) desPro³⁶Exendin-4(1-39)-Lys₆-NH₂ or/and a pharmaceutically acceptable salt thereof, and
(b) metformin or/and a pharmaceutically acceptable salt thereof,
   to a subject in need thereof.

The compounds of (a) and (b) may be administered to a subject in need thereof, in an amount sufficient to induce a therapeutic effect.

The compound desPro³⁶Exendin-4(1-39)-Lys₆-NH₂ (AVE0010, lixisenatide) is a derivative of Exendin-4. AVE0010 is disclosed as SEQ ID NO:93 in WO 01/04156:

SEQ ID NO: 1 AVE0010 (44 AS)
H-G-E-G-T-F-T-S-D-L-S-K-Q-M-E-E-E-A-V-R-L-F-I-E-W-
L-K-N-G-G-P-S-S-G-A-P-P-S-K-K-K-K-K-K-NH₂
SEQ ID NO: 2 Exendin-4 (39 AS)
H-G-E-G-T-F-T-S-D-L-S-K-Q-M-E-E-E-A-V-R-L-F-I-E-W-
L-K-N-G-G-P-S-S-G-A-P-P-P-S-NH₂

Exendins are a group of peptides which can lower blood glucose concentration. The Exendin analogue AVE0010 is characterised by C-terminal truncation of the native Exendin-4 sequence. AVE0010 comprises six C-terminal lysine residues not present in Exendin-4.

In the context of the present invention, AVE0010 includes pharmaceutically acceptable salts thereof. The person skilled in the art knows pharmaceutically acceptable salts of AVE0010. A preferred pharmaceutically acceptable salt of AVE0010 employed in the present invention is acetate.

AVE0010 (desPro³⁶Exendin-4(1-39)-Lys₆-NH₂) or/and a pharmaceutically acceptable salt thereof may be administered by subcutaneous injection. Suitable injection devices, for instance the so-called “pens” comprising a cartridge comprising the active ingredient, and an injection needle, are known. AVE0010 or/and a pharmaceutically acceptable salt thereof may be administered in a suitable amount, for instance in an amount in the range of 10 to 15 μg per dose or 15 to 20 μg per dose once a day (progressive titration from 10 to 15 and to 20 μg/day. 20 μg is the effective maintenance dose).

In the present invention, AVE0010 or/and a pharmaceutically acceptable salt thereof may be administered in a daily dose in the range of 10 to 15 μg or in the range of 15 to 20 μg once a day (progressive titration from 10 to 15 and to 20 μg/day. 20 μg is the effective maintenance dose). AVE0010 or/and a pharmaceutically acceptable salt thereof may be administered by one injection per day.

In the present invention, a liquid composition comprising desPro³⁶Exendin-4(1-39)-Lys₆-NH₂ or/and a pharmaceutically acceptable salt thereof may be employed. The skilled person knows liquid compositions of AVE0010 suitable for parenteral administration. A liquid composition of the present invention may have an acidic or a physiologic pH. An acidic pH preferably is in the range of pH 1-6.8, pH 3.5-6.8, or pH 3.5-5. A physiologic pH preferably is in the range of pH 2.5-8.5, pH 4.0 to 8.5, or pH 6.0 to 8.5. The pH may be adjusted by a pharmaceutically acceptable diluted acid (typically HCl) or pharmaceutically acceptable diluted base (typically NaOH). The preferred pH is in the range of pH 3.5 to 5.0.

The liquid composition may contain a buffer, such as a phosphate, a citrate, an acetate. Preferably, it can contain an acetate buffer, in quantities up to 5 μg/mL, up to 4 μg/mL or up to 2 μg/mL.

The liquid composition of the present invention may comprise a suitable preservative. A suitable preservative may be selected from phenol, m-cresol, benzyl alcohol and p-hydroxybenzoic acid ester. A preferred preservative is m-cresol.

The liquid composition of the present invention may comprise a tonicity agent. A suitable tonicity agent may be selected from glycerol, lactose, sorbitol, mannitol, glucose, NaCl, calcium or magnesium containing compounds such as CaCl₂. The concentration of glycerol, lactose, sorbitol, mannitol and glucose may be in the range of 100-250 mM. The concentration of NaCl may be up to 150 mM. A preferred tonicity agent is glycerol.

In addition, the liquid composition may contain L-methionin from 0.5 μg/mL to 20 μg/mL, preferably from 1 μg/mL to 5 μg/mL. Preferably, it contains L-methionin.

Metformin is the international non proprietary name of 1,1-dimethylbiguanide (CAS Number 657-24-9). In the present invention, the term “metformin” includes any pharmaceutically acceptable salt thereof.

In the present invention, metformin may be administered orally. The skilled person knows formulations of metformin suitable for treatment of diabetes type 2 by oral administration. Metformin may be administered in a dose of at least 1.0 g/day or at least 1.5 g/day. For oral administration, metformin may be formulated in a solid dosage form, such as a tablet or pill.

In the present invention, desPro³⁶Exendin-4(1-39)-Lys₆-NH₂ or/and a pharmaceutically acceptable salt is administered in an add-on therapy to administration of metformin.

In the present invention, the terms “add-on”, “add-on treatment” and “add-on therapy” relate to treatment of diabetes mellitus type 2 with metformin and AVE0010. Metformin and AVE0010 may be administered within a time interval of 24 h. Metformin and AVE0010 each may be administered in a once-a-day-dosage. Metformin and AVE0010 may be administered by different administration routes. Metformin may be administered orally, and AVE0010 may be administered subcutaneously.

The subject to be treated by the method of the present invention suffering from diabetes type 2 may be an obese subject. In the present invention, an obese subject may have a body mass index of at least 30.

The subject to be treated by the method of the present invention may have a HbA1 c value in the range of 7% to 10%.

The subject to be treated by the method of the present invention may be an adult subject. The subject may have an age in the range of 18 to 50 years.

The method of the present invention preferably is a method of treatment of a subject suffering from diabetes type 2, wherein diabetes type 2 is not adequately controlled by treatment with metformin alone, for instance with a dose of at least 1.0 g/day metformin or at least 1.5 g/day metformin for 3 months. In the present invention, a subject the diabetes type 2 of which is not adequately controlled may have a HbA1c value in the range of 7% to 10%.

Another aspect of the present invention is a pharmaceutical combination comprising
(a) desPro³⁶Exendin-4(1-39)-Lys₆-NH₂ or/and a pharmaceutically acceptable salt thereof, and
(b) metformin or/and a pharmaceutically acceptable salt thereof.

Preferably, the combination of the present invention is for treatment of diabetes mellitus type 2.

The combination of the present invention may be administered as described herein in the context of the method of the present invention. The compounds (a) and (b) of the combination of the present invention may be formulated as described herein in the context of the method of the present invention.

Yet another aspect of the present invention is the use of a combination comprising
(a) desPro³⁶Exendin-4(1-39)-Lys₆-NH₂ or/and a pharmaceutically acceptable salt thereof, and
(b) metformin or/and a pharmaceutically acceptable salt thereof,
   for the production of a medicament for the treatment of diabetes mellitus type 2.

The medicament comprises desPro³⁶Exendin-4(1-39)-Lys₆-NH₂ and metformin in separate formulations, as described herein.

The invention is further illustrated by the following example.

### EXAMPLE

24-week study comparing lixisenatide (AVE0010) to sitagliptin as add-on to metformin in obese type 2 diabetic patients younger than 50

Subject of the example is a randomized, double-blind, double-dummy, 2-arm parallel-group, multicenter, 24-week study comparing the efficacy and safety of lixisenatide (AVE0010) to sitagliptin (CAS Number 486460-32-6) as add-on to metformin in obese type 2 diabetic patients younger than 50 years and not adequately controlled with metformin. Sitagliptin is an antidiabetic drug, acting as an inhibitor of dipeptidyl peptidase 4 (DPP4) resulting in enhanced level of Glucagon-Like Peptide 1, thereby reducing blood glucose levels in diabetic patients.

### Study Primary Objectives

The primary objective of this study is to assess the efficacy of lixisenatide on a composite endpoint of glycemic control (HbA1 c) and body weight in comparison to sitagliptin as an add-on treatment to metformin over a period of 24 weeks in obese type 2 diabetic patients younger than 50.

Study Secondary Objectives are assessment of the effects of lixisenatide on:
Absolute changes in HbA1c and body weight
Fasting plasma glucose
Plasma glucose, insulin, C peptide, glucagon and proinsulin during a 2-hour standardized meal test
Insulin resistance assessed by HOMA-IR
Beta cell function assessed by HOMA-beta
To assess lixisenatide safety and tolerability
To assess lixisenatide PK using the population PK approach and to assess anti-lixisenatide antibody development.

### Specific Vulnerable Populations:

Women of child-bearing potential using contraception.

### Inclusion Criteria

Patients (male and female) with type 2 diabetes mellitus, as defined by WHO (21), diagnosed for at least 1 year at the time of screening visit, insufficiently controlled with metformin at a stable dose of at least 1.5 g/day, for at least 3 months prior to the screening visit. Patients with obesity (BMI≧30 kg/m) and aged from 18 years to less than 50 years.

### Exclusion Criteria

HbA1c<7.0% or HbA1c>10% at screening
Type 1 diabetes mellitus
Pregnancy or lactation
Women of childbearing potential with no effective contraceptive method
Fasting Plasma Glucose at screening>250 mg/dL (>13.9 mmol/L).
Weight change of more than 5 kg during the 3 months preceding the screening visit
History of unexplained pancreatitis, chronic pancreatitis, pancreatectomy, stomach/gastric surgery, inflammatory bowel disease
History of metabolic acidosis, including diabetic ketoacidosis within 1 year prior to screening
Hemoglobinopathy or hemolytic anemia or receipt of blood or plasma products within 3 months prior to the time of screening
Within the last 6 months prior to screening: history of myocardial infarction, stroke, or heart failure requiring hospitalization
Known history of drug or alcohol abuse within 6 months prior to the time of screening
Any clinically significant abnormality identified on physical examination, laboratory tests, ECG or vital signs at the time of screening that in the judgment of the investigator or any sub investigator would preclude safe completion of the study or constrains efficacy assessment such as major systemic diseases, presence of clinically significant diabetic retinopathy or presence of macular edema likely to require laser treatment within the study period
Uncontrolled or inadequately controlled hypertension at the time of screening with a resting systolic or diastolic blood pressure>180 mmHg or >110 mmHg, respectively
Laboratory findings at the time of screening:
   Amylase and/or lipase>3 times the upper limit of the normal laboratory range
   Total bilirubin: >1.5 times the upper limit of the normal laboratory range (except in case of Gilbert's syndrome)
   Hemoglobin<11 g/dL and/or neutrophils<1,500/mm³ and/or platelets<100,000/mm³
   Positive test for Hepatitis B surface antigen and/or Hepatitis C antibody
   Positive serum pregnancy test in females of childbearing potential
Use of other oral or injectable antidiabetic or hypoglycemic agents than metformin (e.g., sulfonylurea, alpha glucosidase inhibitor, thiazolidinedione, exenatide, DPP-IV inhibitors, insulin etc.) within 3 months prior to the time of screening
Unstable diet or unstable anti-obesity treatment within 3 months prior to the time of screening
Use of systemic glucocorticoids (excluding topical application or inhaled forms) for one week or more within 3 months prior to the time of screening
Use of any investigational drug within 3 months prior to screening
Clinically relevant history of gastrointestinal disease associated with prolonged nausea and vomiting, including, but not limited to gastroparesis and gastroesophageal reflux disease requiring medical treatment, within 6 months prior to the time of screening
Any previous treatment with lixisenatide (e.g. participation in a previous study with lixisenatide)
Allergic reaction to any GLP 1-agonist in the past (e.g. exenatide, liraglutide) or to metacresol
History of a serious hypersensitivity reaction to sitagliptin.
Moderate or severe renal impairment (creatinine clearance inferior to 50 ml/min)

### Duration of Study Period Per Subject

Maximum duration of 27 weeks±7 days (3-week screening+24-week double-blind, double-dummy, active-controlled treatment+3-day follow-up)

INVESTIGATIONAL PRODUCTS

| | | | Route of |
|---|---|---|---|
| INN | Compound code | Pharmaceutical form | administration |
| Lixisenatide | AVE0010 | injection | subcutaneous |
| Sitagliptin | | capsules | capsules |

STUDY ARMS Number of arms: 2

| Arm Label | Arm description | Arm type |
|---|---|---|
| Lixisenatide | Injection of lixisenatide once a day in the | Experimental |
| | morning within 1 hour prior to breakfast | |
| | (first 2 weeks of double-blind period: | |
| | titration 10 to 15 μg, then 15 to 20 μg) and | |
| | one capsule of sitagliptin placebo intake in | |
| | the morning or without food. On top of | |
| | metformin background therapy. | |
| Sitagliptin | One capsule of sitagliptin intake in the | Active |
| | morning with or without food and | Calibrator/ |
| | lixisenatide matched placebo injection | Comparator |
| | once a day in the morning within hour | |
| | prior to breakfast. On top of metformin | |
| | background therapy. | |

ENDPOINTS

| | Time frame |
|---|---|
| | for evaluation |
| Primary Endpoint(s): | |
| Percentage of patients with HbA1c values <7% | 24 weeks |
| AND a weight loss of at least 5% of baseline body | |
| weight | |
| Secondary Endpoint(s): | |
| Absolute change in HbA1c values | 24 weeks |
| Percentage of patients with HbA1c values ≦6.5% | 24 weeks |
| Absolute change in body weight | 24 weeks |
| Change in fasting plasma glucose | 24 weeks |
| Change in plasma glucose and in β-cell function | 24 weeks |
| during a test meal | |
| Change in insulin resistance assessed by HOMA-IR | 24 weeks |
| Change in β-cell function assessed by HOMA-β | 24 weeks |
| Percentage of patients requiring rescue therapy | 24 weeks |
| during the double-blind period | |

We claim:

1. A method for treating diabetes mellitus type 2 comprising administering
(a) desPro³⁶Exendin-4(1-39)-Lys₆-NH₂ or a pharmaceutically acceptable salt thereof, and
(b) metformin or a pharmaceutically acceptable salt thereof,
to a subject in need thereof.

2. The method according to claim 1, wherein desPro³⁶Exendin-4(1-39)-Lys₆-NH₂ or a pharmaceutically acceptable salt thereof is administered subcutaneously.

3. The method according to claim 1, wherein the metformin is administered orally.

4. The method according to claim 1, wherein desPro³⁶Exendin-4(1-39)-Lys₆-NH₂ or a pharmaceutically acceptable salt is administered in an add-on therapy to administration of metformin.

5. The method according to claim 1, wherein the subject to be treated is obese.

6. The method according to claim 5, wherein the subject has a body mass index of at least 30.

7. The method according to claim 1, wherein the subject to be treated is an adult subject.

8. The method according to claim 1, wherein diabetes mellitus type 2 is not adequately controlled with metformin alone.

9. The method according to claim 8, wherein treatment with a dose of at least 1.5 g/day metformin alone for three months does not adequately control diabetes mellitus type 2.

10. The method according to claim 1, wherein the subject to be treated has a HbA1c value in the range of 7% to 10%.

11. A pharmaceutical combination comprising
(a) desPro³⁶Exendin-4(1-39)-Lys₆-NH₂ or a pharmaceutically acceptable salt thereof, and
(b) metformin or a pharmaceutically acceptable salt thereof.

12. The pharmaceutical combination according to claim 11, wherein the pharmaceutical combination is for treating diabetes mellitus type 2.


SEQUENCE LISTING (2 sequences)

SEQ ID NO: 1 - protein, 44 aa, Artificial
  AVE0010 (44 AS) Position 44 (Lys) is amidated
HGEGTFTSDLSKQMEEEAVRLFLEWLKNGGPSSGAPPSKKKKKK

SEQ ID NO: 2 - protein, 39 aa, Artificial
  Heloderma Suspectum Position 39 (Ser) is amidated
HGEGTFTSDLSKQMEEEAVRLFIEWLKNGGPSSGAPPPS